(19) United States
(12) Patent Application Publication
(10) Pub. No.: US 2023/0194558 A1
(43) Pub. Date: Jun. 22, 2023

(54) END EFFECTOR ASSEMBLIES, SYSTEMS, AND METHODS OF USE

(71) Applicant: Advanced Solutions Life Sciences, LLC, Louisville, KY (US)

(72) Inventors: Rob ELI, Louisville, KY (US); Alex SCHULTZ, Louisville, KY (US); Matt GDOWSKI, Tucson, AZ (US); Justin PALMER, Fort Wayne, IN (US); Scott CAMBRON, Louisville, KY (US)

(21) Appl. No.: 18/084,229

(22) Filed: Dec. 19, 2022

Related U.S. Application Data
(60) Provisional application No. 63/290,798, filed on Dec. 17, 2021.

(51) Int. Cl. G01N 35/10; G01N 35/00; B33Y 30/00; B33Y 50/02; B33Y 10/00

(52) U.S. Cl. CPC G01N 35/1016; G01N 35/0099; G01N 35/00732; B33Y 30/00; B33Y 50/02; B33Y 10/00

(57) ABSTRACT

End effector assemblies according to the present disclosure include a tool body mounted to a robotic arm and an impedance-measuring tip coupled to the tool body. The impedance-measuring tip defines a first volume to receive a fluid and a first dispensing outlet for dispensing the fluid. The impedance-measuring tip includes an impedance-measuring sensor configured to output a signal indicative of a change in impedance. A tip extension is fluidically coupled to the impedance-measuring tip that defines a second volume for receiving the fluid. A camera is coupled to the tool body and configured to capture image data of the second volume that captures at least a visual representation of a number of cells or other objects in the second volume. A pump is coupled to the impedance-measuring tip to dispense the fluid from the first volume into the second volume and from the second volume into a receptacle.

### CROSS REFERENCE TO RELATED APPLICATION

This application claims priority to U.S. Provisional Application Ser. No. 63/290,798 filed on Dec. 17, 2021, the entirety of which is hereby incorporated by reference.

### FIELD

The present disclosure relates generally to end effector assemblies, systems, and methods, and more particularly, to end effector assemblies, systems, and methods that include an impedance-measuring tip to measure precise amounts of target material for dispensing onto a substrate or other receptacle.

### BACKGROUND

Three-dimensional printers may dispense biological materials, including cells. For certain biological assays, it may be desirable to dispense a controlled number [symbol] for example, a single cell [symbol] onto a substrate, such as within a well, test tube, cuvette, or other suitable receptacle for carrying out the desired assay. Accordingly, it may be desirable to have dispensing tools for three-dimensional printers that provide for dispensing of single cells or other precise amount onto a substrate. Further, it may be desirable that the tool have capability for verifying the deposition of the for quality control purposes.

### SUMMARY

In one embodiment, an end effector assembly is provided. The end effector assembly includes a tool body configured to be mounted to a robotic arm and an impedance-measuring tip coupled to the tool body. The impedance-measuring tip defines a first volume configured to receive a fluid and a first dispensing outlet for dispensing the fluid from the first volume. The impedance-measuring tip includes an impedance-measuring sensor configured to output a signal indicative of a change in impedance of the fluid advanced out of the first dispensing outlet. The change in impedance is indicative of cells or other objects passing from the first volume out of the first dispensing outlet. The end effector assembly also includes a tip extension fluidically coupled to the impedance-measuring tip. The tip extension defines a second volume for receiving the fluid from the first dispensing outlet. A camera is coupled to the tool body and configured to capture image data of the second volume. The image data captures at least a visual representation of the cells or other objects in the second volume. A pump is coupled to the impedance-measuring tip and operable to dispense the fluid from the first volume into the second volume and from the second volume into a receptacle via a second dispensing opening formed in the tip extension.

In another embodiment, a dispensing tool system is provided. The dispensing tool system includes a tool body configured to be mounted to a robotic arm and an impedance-measuring tip coupled to the tool body. The impedance-measuring tip defines a first volume configured to receive a fluid and a first dispensing outlet for dispensing the fluid from the first volume. The impedance-measuring tip includes an impedance-measuring sensor configured to output a signal indicative of a change in impedance of the fluid advanced out of the first dispensing outlet. The change in impedance is indicative of cells or other objects passing from the first volume out of the first dispensing outlet. A tip extension is fluidically coupled to the impedance-measuring tip; the tip extension defines a second volume for receiving the fluid from the first dispensing outlet. A camera is coupled to the tool body and configured to capture image data of the second volume. The image data captures at least a visual representation of the cells or other objects in the second volume. The system also includes a print stage including a substrate, the substrate including a receptacle that receives the fluid in the second volume. A second camera is coupled to the print stage and configured to capture the image data of the second volume. A pump is coupled to the impedance-measuring tip and operable to dispense the fluid from the first volume into the second volume and from the second volume into the receptacle via a second dispensing opening formed in the tip extension.

In yet another embodiment, a method for dispensing cells is provided. The method for dispensing cells includes submerging at least a portion of a tip extension fluidically coupled to an impedance-measuring tip into a fluid reservoir. The impedance-measuring tip defines a first volume configured to receive a fluid and a first dispensing outlet for dispensing the fluid from the first volume. The impedance-measuring tip includes an impedance-measuring sensor configured to output a signal indicative of a change in impedance of the fluid advanced out of the first dispensing outlet, the change in impedance being indicative of cells or other objects passing from the first volume out of the first dispensing outlet. The tip extension defines a second volume. The method further includes aspirating a first volume of fluid into the first volume of the impedance-measuring tip through the tip extension, dispensing a portion of the first volume of fluid from the first dispensing outlet into the second volume of the tip extension as a second volume of fluid, measuring the impedance of the fluid advanced out of the first dispensing outlet and into the second volume with the impedance-measuring sensor, determining with a controller communicatively coupled to the impedance-measuring sensor, a number of cells within the second volume of fluid, translating the impedance-measuring tip over a print stage, and dispensing the second volume of fluid from the second volume into a receptacle when the numbers of cells within the second fluid volume matches a desired number of cells.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an end effector assembly, according to one or more embodiments illustrated and described herein;

FIG. 2A schematically depicts a tip extension uncoupled from the impedance-measuring tip of the end effector assembly, according to one or more embodiments illustrated and described herein;

FIG. 2B schematically depicts a cross-section of the tip extension as coupled to the impedance-measuring tip of FIG. 2A, according to one or more embodiments shown and described herein

FIG. 2C schematically illustrates a modified version of the tip extension coupled to an impedance-measuring tip, according to one or more embodiments shown and described therein;

FIG. 3 schematically depicts a system including a controller for operating various components of the end effector assembly and/or a dispensing tool system, according to one or more embodiments illustrated and described herein;

FIG. 4 schematically depicts a dispensing tool system, according to one or more embodiments illustrated and described herein;

FIG. 5A schematically depicts an air knife with the tip extension inserted into an air knife aperture, according to one or more embodiments illustrated and described therein;

FIG. 5B schematically depicts a cross-section of the air knife, according to one or more embodiments illustrated and described herein;

FIG. 5C schematically depicts a stage light fixture, according to one or more embodiments shown and described herein;

FIG. 6A schematically depicts a tip extension with a impedance-measuring tip coupled thereto inserted into a fluid reservoir, according to one or more embodiments illustrated and described herein;

FIG. 6B schematically depicts aspiration of working fluid into the tip extension and the impedance-measuring tip coupled, according to one or more embodiments illustrated and described herein;

FIG. 6C schematically depicts a first fluid volume in the impedance-measuring tip and a second fluid volume in the tip extension, according to one or more embodiments shown and described herein; and

FIG. 6D depicts release of the second fluid volume from the tip extension, according to one or more embodiments shown and described herein.

### DETAILED DESCRIPTION

The present disclosure is directed to an end effector assembly for accurate dispensing of cells or other particles onto a substrate. End effector assemblies, dispensing tool systems, and methods for dispensing cells or other particles as described herein have the ability to dispense a single cell onto the substrate. While single cell dispensing onto the substrate is one of the contemplated applications of the present disclosure, other applications of the end effector assemblies, dispensing tool systems, and methods for dispensing cells are contemplated and possible. For example, dispensing multiple cells or other type particles.

End effector assemblies according to various embodiments of the present disclosure may include a tool body, an impedance-measuring tip, and a tip extension coupled to the impedance-measuring tip. The impedance-measuring tip includes an impedance-measuring sensor configured to output a signal indicative of a change in impedance of a fluid being passed from a first volume of the impedance-measuring tip to a second volume of the tip extension. As noted above, the signal may be indicative of cells or other objects passing from the first volume of the impedance-measuring tip to the second volume of the tip extension, though other indications (e.g., material type are contemplated and possible). In embodiments, the end effector assembly may further include a camera configured to capture image data of the second volume within the tip extension. The image data may include a visual representation of the cells or other objects located within the tip extension. Accordingly, output of the signal from the impedance-measuring sensor can be confirmed based on the image data. Accordingly, accurate dispensing of a desired number of cells, cell material, or the like can be reliably achieved. These and additional embodiments will be described in greater detail herein.

As used throughout the present disclosure, the terms [symbol] upstream [symbol] and [symbol] downstream [symbol] refer to the relative positioning of unit operations with respect to the direction of flow of the process streams out of the tip extension and/or the impedance-measuring tip. A first unit operation of a system may be considered [symbol] upstream [symbol] of a second unit operation if process streams flowing through the system encounter the first unit operation before encountering the second unit operation Likewise, a second unit operation may be considered [symbol] downstream [symbol] of the first unit operation if the process streams flowing through the system encounter the first unit operation before encountering the second unit operation.

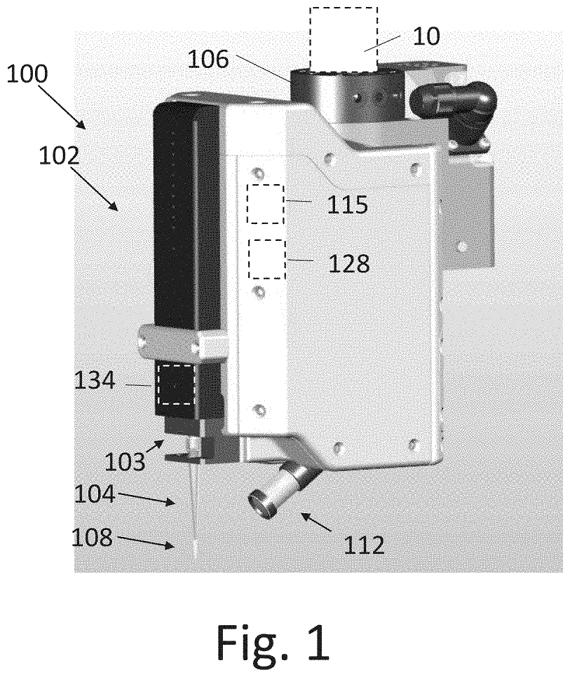

Referring now to FIG. 1, an end effector assembly 100 may generally include a tool body 102, impedance-measuring tip 104, a tip extension 108, a pump 115, a first camera 112, and a controller 128. A greater or fewer number of components may be included without departing from the scope of the present disclosure. As will be described in greater detail below, end effector assemblies as described herein may be incorporated into robotic print or dispensing systems, such as BioAssemblyBot® 3-D printing and robotics systems such as described in U.S. patent application Ser. No. 15/726,617, filed Oct. 6, 2017, entitled [symbol] System and Method for a Quick-Change Material Turret in a Robotic Fabrication and Assembly Platform, [symbol] hereby incorporated by reference in its entirety and as available from Advanced Solutions Life Sciences, LLC of Louisville, Ky.

The tool body 102 may be configured to be mounted to a robotic arm 10 (e.g., a six axis robotic arm, a four axis robotic arm, a two axis robotic arm, etc.). For example, the tool body 102 may comprise a mounting portion 106 configured to be rigidly coupled to the robotic arm 10 via any type of fastener or connector. The tool body 102 may be formed of any material (e.g., metal, plastic, etc.) capable of supporting the various components described here. In some embodiments, the tool body 102 is a shell within which various components (e.g., the pump 115, the controller 128, or other components) may be housed. In embodiments, the tool body 102 may define a tool aperture 103 for receiving and coupling to the impedance-measuring tip 104. In embodiments, the tool aperture 103 may also facilitate connection (e.g., communicative coupling or fluidic coupling) of the impedance-measuring tip 104 with the controller 128 and/or the pump 115.

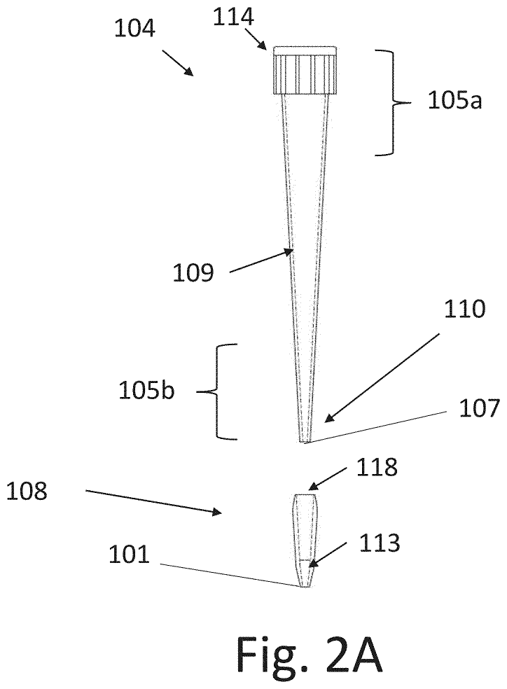

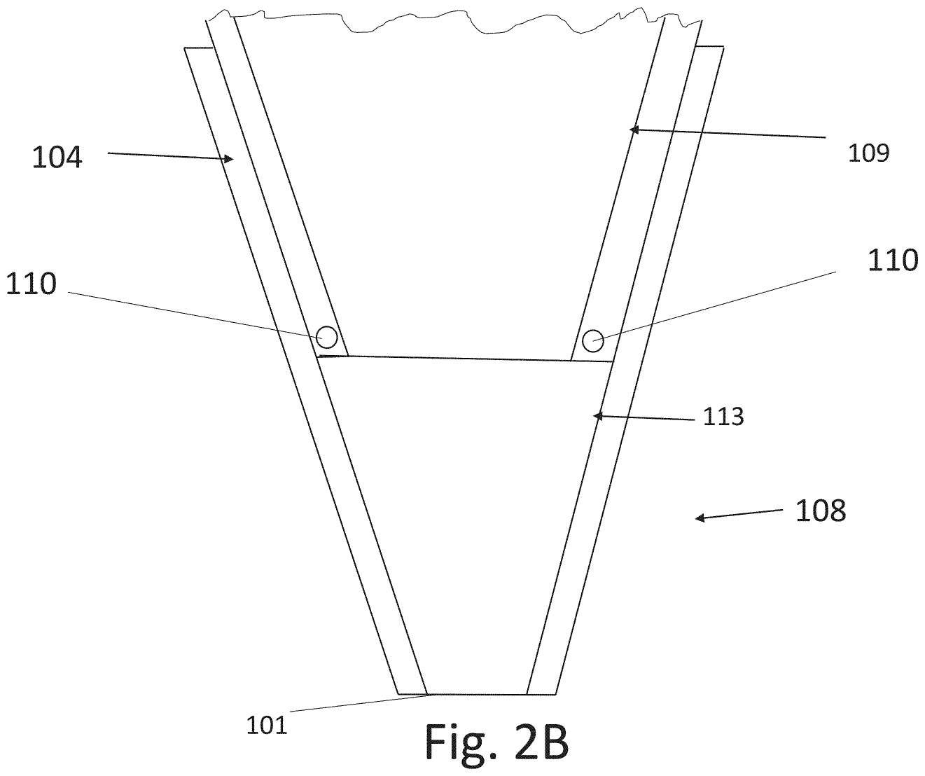

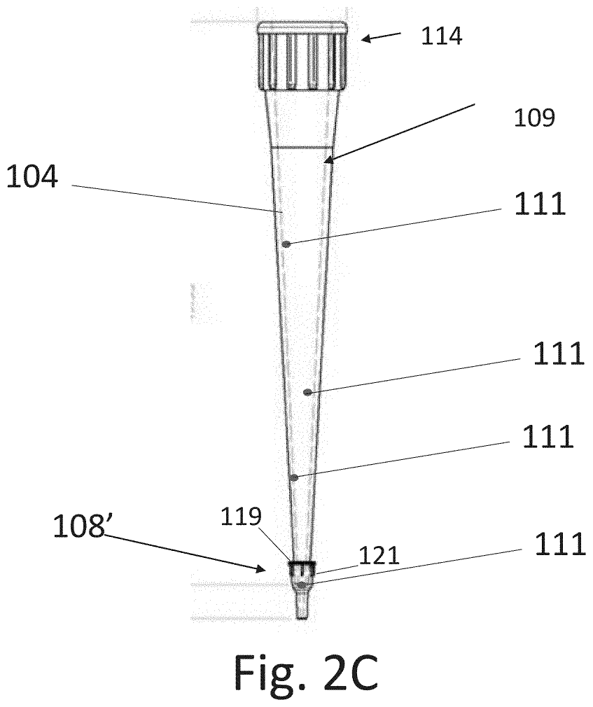

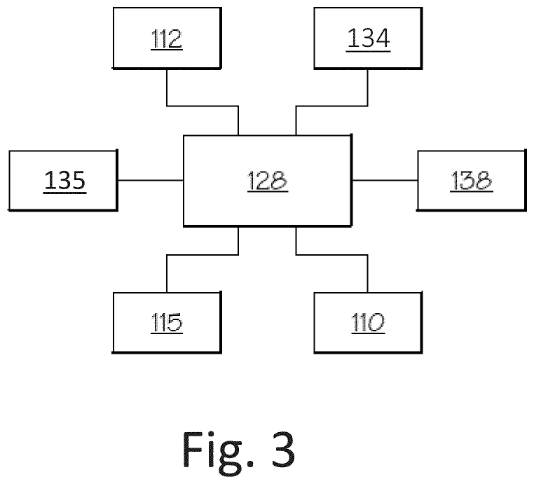

The impedance-measuring tip 104 is coupled to the tool body 102, and may be removably coupled to the tool body 102 such that the impedance-measuring tip 104 may be replaced. For example, the impedance-measuring tip 104 may be inserted into the tool aperture 103 to couple the impedance-measuring tip 104 to the tool body 102. The impedance-measuring tip 104 is further illustrated with respect to FIG. 2A-2C. In embodiments, and as more fully illustrated in FIG. 2A, the impedance-measuring tip 104 may include a mounting end 105a and a dispensing end 105b opposite the mounting end 105a. In embodiments, the mounting end 105a may include a fitting 114 configured to mount the mounting end 105a to the tool body 102 (e.g., via push fit, clip connections, fastener connections, etc.). In embodiments, the fitting 114 may include electrical and/or fluid pathways for coupling the impedance-measuring tip 104 to the pump 115 (e.g., via fluidic coupling) and the controller 128 (e.g., via electrical coupling, though wireless communication is contemplated and possible). The impedance-measuring tip 104 may define a first volume 109 configured to receive a fluid (e.g., cell culture media with cells or other suitable fluid) and house a first fluid volume. The first volume 109 may be capable of holding up to about 10 microliters of fluid, up to about 50 microliters of fluid, up to about 100 microliters of fluid, up to about 200 microliters of fluid, up to about 400 microliters of fluid, up to about 600 microliters of fluid, up to about 800 microliters of fluid, up to about 1,000 microliters of fluid, or any other suitable volume. At the dispensing end 105b a first dispensing outlet 107 may be formed for aspiration and dispensing of fluid. The impedance-measuring tip 104 may be formed of any combination of materials (e.g., polyethylene, polypropylene, glass, metals, a combination thereof, etc.). It is noted that in some embodiments, the impedance-measuring tip 104 may be substantially transparent or translucent such as about the first volume 109 to allow visual pathways into the first volume 109.

In various embodiments, impedance-measuring tip 104 may be disposable and/or reusable. In embodiments, the impedance-measuring tip 104 may be used to carry out at least about 10, at least about 50, at least about 100, at least about 110, at least about 120, at least about 130, at least about 140, or at least about 150 dispense events. For example, dispense events include dispensing of a fluid such as noted above through the first dispensing outlet 107.

FIG. 2D illustrates a schematic cross-section of the dispensing end 105b of the impedance-measuring tip 104 and the extension member. As illustrated The impedance-measuring tip 104 includes an impedance-measuring sensor 110 configured to output a signal indicative of a change in impedance of the fluid as it is advanced out of the first dispensing outlet 107. That is, the impedance-measuring sensor 110 may monitor, such as in real- or near real-time, a change in impedance of the fluid as the fluid is dispensed out of the first dispensing outlet 107. For example, the impedance-measuring sensor 110 may include an impedance meter, electrodes utilizing electrochemical impedance spectroscopy, or any other suitable device for measuring impedance. The impedance-measuring sensor 110 may be positioned at or adjacent to first dispensing outlet 107 to measure the change in impedance of the fluid just before or as it exits the first dispensing outlet 107 from the first volume 109. The impedance-measuring sensor 110 may measure impedance or changes in impedance ranging from about 20 kiloohms to about 70 kiloohms, for example, though other ranges are contemplated and possible. As a change in impedance within the fluid is detected, such signal may be indicative of cells or other objects passing over the impedance-measuring sensor 110 toward or out of the first dispensing outlet 107. Accordingly, the signal output by the impedance-measuring sensor 110 allows the end effector assembly 100 to function as a Coulter counter. That is, based on the output signal of the impedance-measuring sensor 110, the controller 128 may count and/or size a single cell 111 as it passes from the impedance-measuring tip 104, to allow for precise control of dispensing parameters. Accordingly, the impedance-measuring tip 104 may confirm clonality of a dispensed amount based on a measured impedance profile.

Still referring to FIGS. 2A-2C, to prevent accidental dispensing of fluid with unwanted cell counts (e.g., too high or too small), coupled, such as fluidically coupled, to the impedance-measuring tip 104 is the tip extension 108. For example, the tip extension 108 is positioned downstream of the impedance-measuring tip 104 so as to receive fluid from the first dispensing outlet 107. The tip extension 108 defines a second volume 113, best illustrated in FIG. 2B for receiving fluid from the impedance-measuring tip 104. The second volume 113 may be capable of holding a second fluid volume, which may be up to about 0.5 microliters of fluid, up to about 1.0 microliters of fluid, up to about 2.0 microliters of fluid, up to about 3.0 microliters of fluid, up to about 4.0 microliters of fluid, up to about 5.0 microliters of fluid, up to about 10 microliters of fluid, or any other suitable volume. As illustrated, the tip extension 108 may define a receiving aperture 118 fluidically coupled to the second volume 113 for receiving the dispensing end 105b of the impedance-measuring tip 104, such that the first dispensing outlet 107 is positioned within the second volume 113 when assembled. The tip extension 108 may further define a second dispensing outlet 101 for aspiration of fluid into and/or dispensing of fluid out of the second volume 113.

The tip extension 108 may be made of polyethylene, polypropylene, polycarbonate, cyclic olefin copolymer, metals, a combination thereof, or any other suitable material. In embodiments, the tip extension 108 may be substantially translucent or transparent to allow for visual pathways into the second volume 113. The tip extension 108 may be disposable and/or reusable. In some embodiments, the tip extension 108 may be reused to carry out at least about 10, at least about 50, at least about 100, at least about 110, at least about 120, at least about 130, at least about 140, or at least about 150 dispense events.

With reference particularly to FIG. 2A in embodiments, the tip extension 108 may be removably coupled to the dispensing end 105b of the impedance-measuring tip 104. For example, the tip extension 108 may be press-fit onto the dispensing end 105b of the impedance-measuring tip 104 (such as via a slip lock press fit coupling), threadingly coupled to the dispensing end 105b of the impedance-measuring tip 104, or the like. It is noted that in some embodiments, the impedance-measuring sensor 110 may be positioned within the tip extension 108 when the impedance-measuring tip 104 is coupled to the tip extension 108. In embodiments, it is contemplated that the impedance-measuring sensor 110 could instead be coupled to the tip extension 108 to measure fluid as it enters the second volume 113.

In various embodiments, the tip extension 108 may seal to the impedance-measuring tip 104. For example, the tip extension 108 may have a sealing valve or the like, such that fluid does not leak between the tip extension 108 and the impedance-measuring tip 104.

FIG. 2C illustrates modified embodiment of the tip extension 108 [symbol] . That is the tip extension 108 [symbol] illustrated in FIG. 2C is substantially identical to the tip extension 108 described above. However, in the present embodiment, the tip extension 108 [symbol] includes a flange 119 and/or a plurality of ribs 121 extending radially from the tip extension 108 [symbol] . The flange 119 and/or the plurality of ribs 121 may assist with gripping the tip extension 108 [symbol] by a user to mount or remove the tip extension 108 [symbol] from the impedance-measuring tip 104.

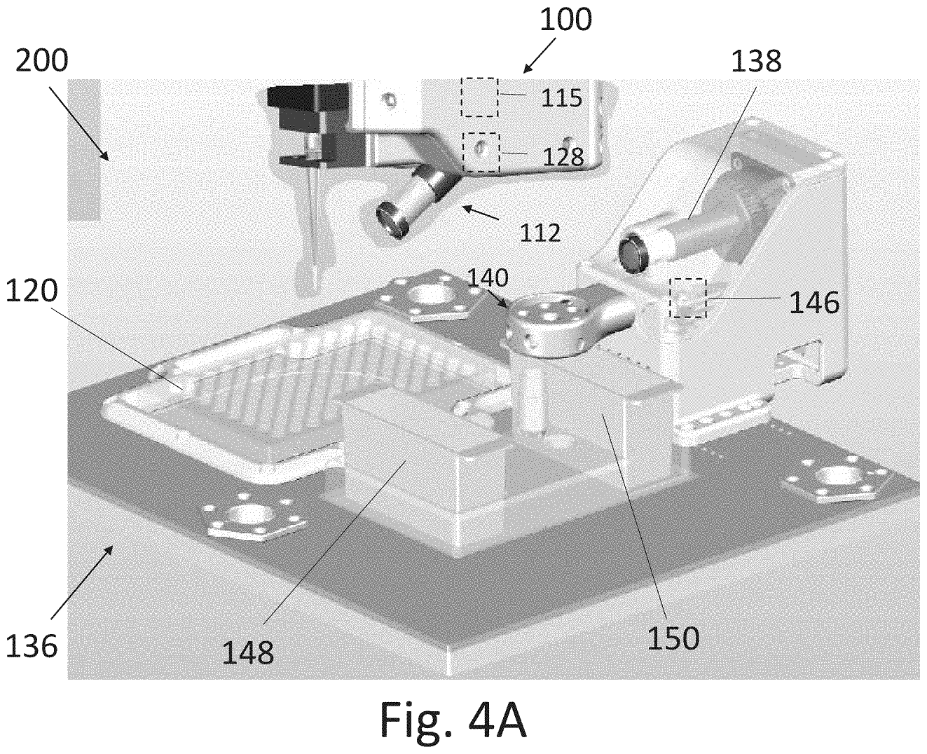

As noted above, and with reference to FIG. 1 the end effector assembly 100 may include a controller 128. For example, the controller 128 may include any number of chips, memories, processors, etc. to allow the controller 128 to execute non-transitory computer readable instructions to operate various functionality of the end effector assembly 100 as will be described in greater detail below. Though the controller 128 is schematically illustrated as being coupled to the tool body 102, it need not be. In embodiments, the controller 128 may operate in a distributed computing environment such that one or more portions (e.g., processors, memories, chipsets, etc.) are communicatively coupled to one another wirelessly (e.g., via a network, cloud computing etc.) and may be positioned remotely from the end effector assembly 100. In embodiments, the controller 128 is communicatively coupled (e.g., via wires or wirelessly) to the impedance-measuring sensor 110. Accordingly, the controller 128 executing non-transitory computer readable instructions, may receive the signal from the impedance-measuring sensor 110, and process the signal to determine a number of cells or other objects that has been dispensed or is being dispensed into the second volume 113 from the first volume 109. That is, the controller 128 with the impedance-measuring sensor 110 may function as a Coulter counter and may count and/or size a single cell 111 as it passes from the first dispensing outlet 107 to the second volume 113 of the tip extension 108. That is, based on the signal from the impedance-measuring sensor 110, the controller 128 may be configured to determine a number and/or size characteristics of a cell passing thereby. In embodiments, as will be described in greater detail below, the controller 128 may control dispensing and/or aspiration of fluid based on the signal from the impedance-measuring sensor 110. For example, in embodiments, where it is determined that there is a single cell within the second volume 113, the controller 128 may operate to dispense the single cell 111 onto a substrate, within an awaiting well or petri-dish, or the like. However, it is further noted, that in embodiments where a greater number of cells are desired, the controller 128 may execute machine-readable instructions to determine a desired number of cells (e.g., two, three, four, etc.) are within the second volume 113 prior to dispensing the cells. In embodiments, if it is determined that an unwanted number of cells are positioned within the second volume 113 of the tip extension 108, the fluid within the second volume 113 may be disposed of, such as within a waste receptacle 150 (schematically depicted in FIG. 4).

In some embodiments, the controller 128 may execute non-transitory computer readable instructions to detect cells of differing biological materials or other objects. For example, cells of a first material may have a different impedance value than cells of a second material. For example, particular bacterial strains may have known impedance values (e.g., 30 kiloohms). Accordingly, the controller 128 may determine whether the particular bacterial strain is present in the second volume 113. As another example, a hepatocyte cell may have a known impedance value of 50 kiloohms. Accordingly, the controller 128 may determine whether the hepatocyte cell is present. Based on the presence of the particular biological material, the controller 128 may further cause the end effector assembly 100 to dispense the second fluid volume within the waste receptacle 150 or on a substrate, well, petri-dish, or the like).

In embodiments, the controller 128, via artificial intelligence and machine-learning, may be trained (such as via training with a neural network one or more identification models) to identify a cell or particle, a number of cells or particles, a size of the cell(s) or particle(s), a material type (e.g., bacteria, cell type, organoid, etc.), or the like passing out of the first dispensing outlet 107 based on the change in impedance. For example, a model may be trained on training data including measured impedance or changes in impedance to determine cells and/or particles, numbers of cells and/or particles, size of cells and/or particles, materials types of cells and/or particles, etc. to correlate a measured change in impedance to any one of the above characteristics.

In embodiments, the controller 128 may be communicatively coupled to the robotic arm 10 so as to move the end effector assembly 100 with the robotic arm 10 as desired, for dispensing cells, waste disposal, or the like.

Still referring to FIG. 1, as noted above, in embodiments, the end effector assembly 100 may include a first camera 112. The first camera 112 may be a high-powered microscope camera coupled to the tool body 102 and capable of imaging individual cells. In embodiments, the first camera 112 may be able to zoom as needed for clearer image data. As illustrated in FIG. 1, the first camera 112 may be positioned on the tool body 102 to face the dispensing end 105b of the impedance-measuring tip 104 and the tip extension 108. For example, the first camera 112 may be angled to particularly capture image data of the second volume 113 of the tip extension 108. However, it is contemplated that the first camera 112 may capture image data of cells or other objects present in the first volume 109 and/or the second volume 113. The first camera 112 may be communicatively coupled to the controller 128, which may execute instructions control the first camera 112 (e.g., a level of zoom of the first camera 112) and/or to determine a number of cells within the first volume 109 and/or the second volume 113 (e.g., such as via object recognition software). In embodiments, the image data may be video data. In some embodiments, the controller 128 may determine the number of cells in the first volume 109 and/or the second volume 113 in real- or near-real-time. In other embodiments, it is contemplated that the first camera 112 (in addition to or in lieu of communication with the controller 128), may provide the image data (such as via a user interface device 135, display, or the like) to a user to allow the user to confirm the number of cells in the image data.

As noted above, a pump 115 (or another type actuator) may be operatively coupled to the impedance-measuring tip 104. In some embodiments, the pump 115 may include both a vacuum pump and a positive pressure pump. In embodiments, the pump 115 may also be communicatively coupled to the controller 128, such that the controller 128 may execute instructions to control operation of the pump 115 to aspirate and/or dispense fluid. For example, during initial filling of the impedance-measuring tip 104, the pump 115 may be actuated by the controller 128 to aspirate a fluid into the first volume 109. Subsequently, the pump 115 may be actuated by the controller 128 to dispense fluid from the first volume 109 into the second volume 113 via the first dispensing outlet 107 and/or from the second volume 113 into a receptacle 120 via the second dispensing outlet 101. The pump 115 may be a pressure-driven pump that allows for discrete flow control through the impedance-measuring tip 104 and the tip extension 108. Pressures for aspirating fluid may be about [symbol] 10 millibar, about [symbol] 20 millibar, about [symbol] 30 millibar, about [symbol] 50 millibar, about [symbol] 80 millibar, about [symbol] 100 millibar, about [symbol] 120 millibar, about [symbol] 150 millibar, about [symbol] 200 millibar, or any other suitable pressure. Pressures for dispensing fluid may be about 1.0 millibar, about 5.0 millibar, about 10 millibar, about 15 millibar, about 20 millibar, about 25 millibar, about 30 millibar, or any other suitable pressure. In embodiments, the controller 128 may control dispensing parameters (e.g., pressure/time) depending on a particular application, cellular material detects, etc.

In various embodiments, the controller 128 may store a number of cells dispensed within a memory of the controller 128 or other memory (e.g., remote server, external hard drive, etc.) communicatively coupled to the controller 128. Accordingly, in embodiments, the memory may be accessed to determine a number of cells dispensed for a particular dispensing event. For example, a dispensing event may be stored with an identifier identifying the particular dispensing event and saved for later recall, data analytics, or the like.

FIG. 4 schematically depicts the controller 128 communicatively coupled via a communication path (e.g., any number of buses, waveguides, wires, or wireless mediums) to the first camera 112, the pump 115, and the impedance-measuring sensor 110. In embodiments, the controller 128 may be further be communicatively coupled to the robotic arm 10 such as noted above, a second camera 138, a user interface device 135, an air knife 140, and/or a stage light fixture 146, each of which may form part of a dispensing tool system 200 described further below.

In embodiments, a user interface device 135 may include any device allowing a user to receive information from the controller 128 and/or interact with the controller 128 to input instructions. For example, the user interface device 135 may include a display for displaying image data to the user. The user interface device 135 may include any number of input devices such as a touch screen, buttons, toggles, switches, keyboards, etc. allowing a user to provide instructions for operation of the robot end effector, or the dispensing tool system 200 later described. For example, in embodiments, the user may input instructions to the controller 128 with respect to number of cells to dispense in a single dispense event, cell material type to dispense, etc.

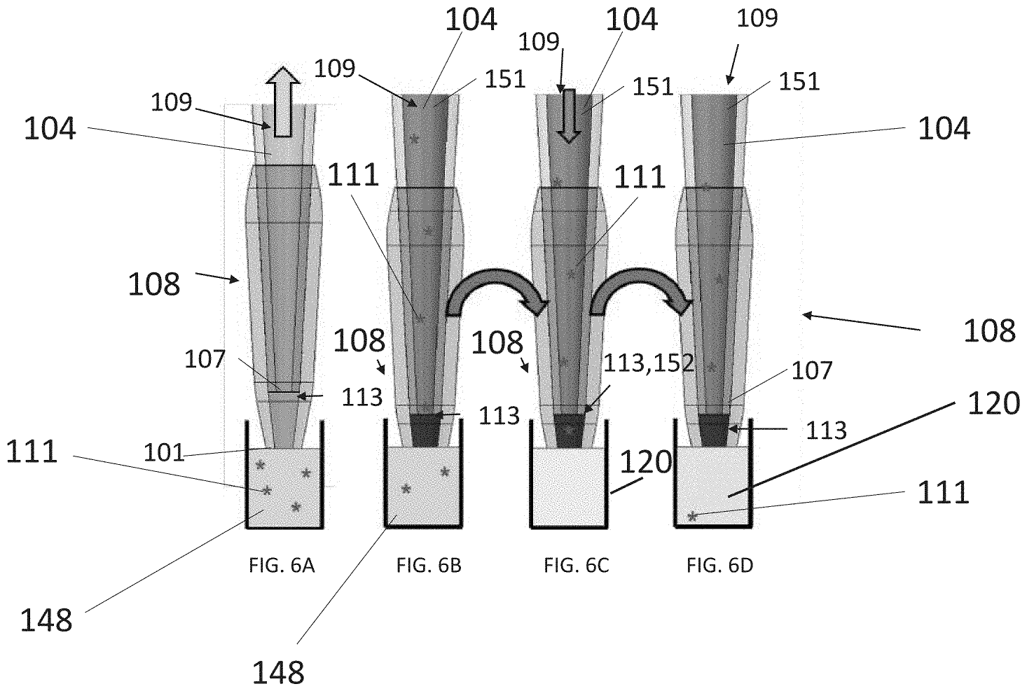

FIG. 6 schematically illustrates a dispensing tool system 200. The dispensing tool system 200 may include the end effector assembly 100 described above, coupled to a robotic arm 10 (not shown) so as to be positioned above a print stage 136. As noted above, the dispensing tool system 200 may further include the second camera 138, the user interface device 135, the air knife 140, and/or a stage light fixture 146. A greater or fewer number of components may be included without departing from the scope of the present disclosure.

The print stage 136 may include a receptacle 120. The receptacle 120 may include a petri dish, a single-well plate, a multi-well plate, a tray, a bottle, a vial, a cuvette, or any other suitable receptacle for carrying out a desired assay (e.g., receiving fluid dispensed from the second volume 113 of the tip extension 108 during the dispense event). In embodiments, the print stage 136 may be rotatable or movable and/or more be controllable by the controller 128. The controller 128 may be communicatively coupled to the robotic arm 10 to move the end effector assembly 100 to various positions about the print stage 136 such as to the receptacle 120, to a fluid reservoir 148, to the waste receptacle 150, and/or to the air knife 140.

The fluid reservoir 148, the waste receptacle 150, and/or the air knife 140, may be positioned on or adjacent the print stage 136 for convenient access. The fluid reservoir 148 may hold fluid (e.g., cell-laden fluid/medium) to be aspirated by the end effector assembly 100. The waste receptacle 150 may be used when an undesirable number of cells and/or biological material is positioned within the second volume 113 of the tip extension 108.

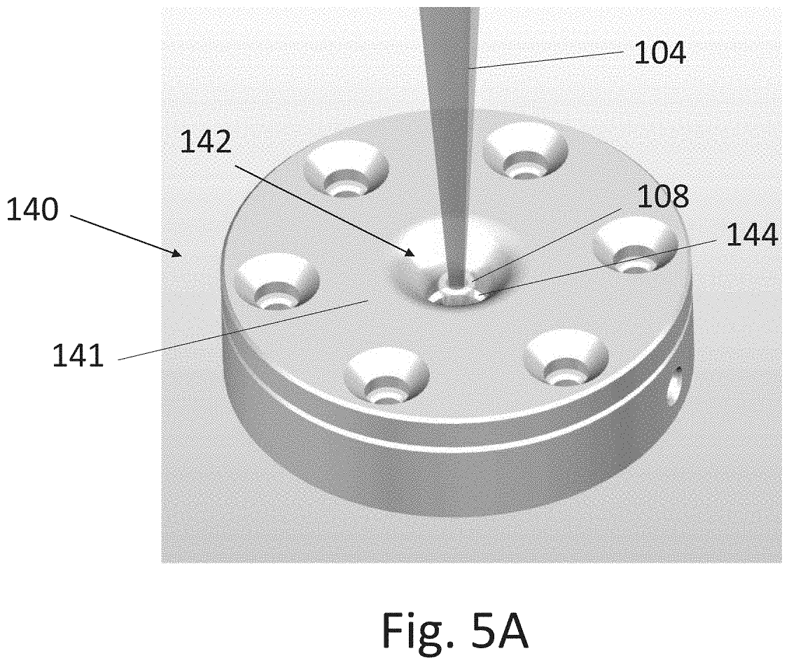

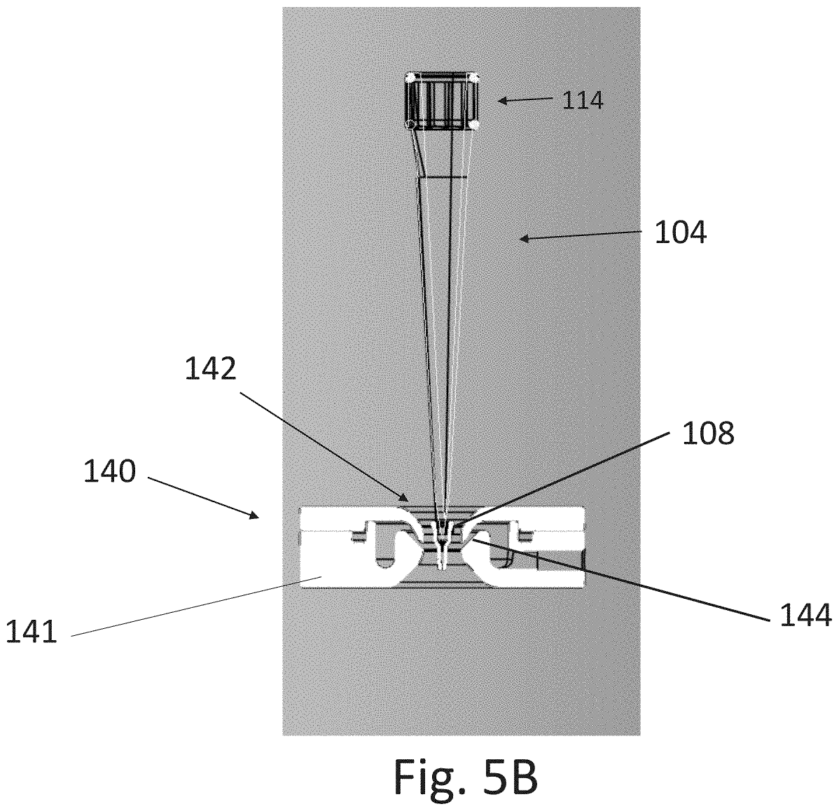

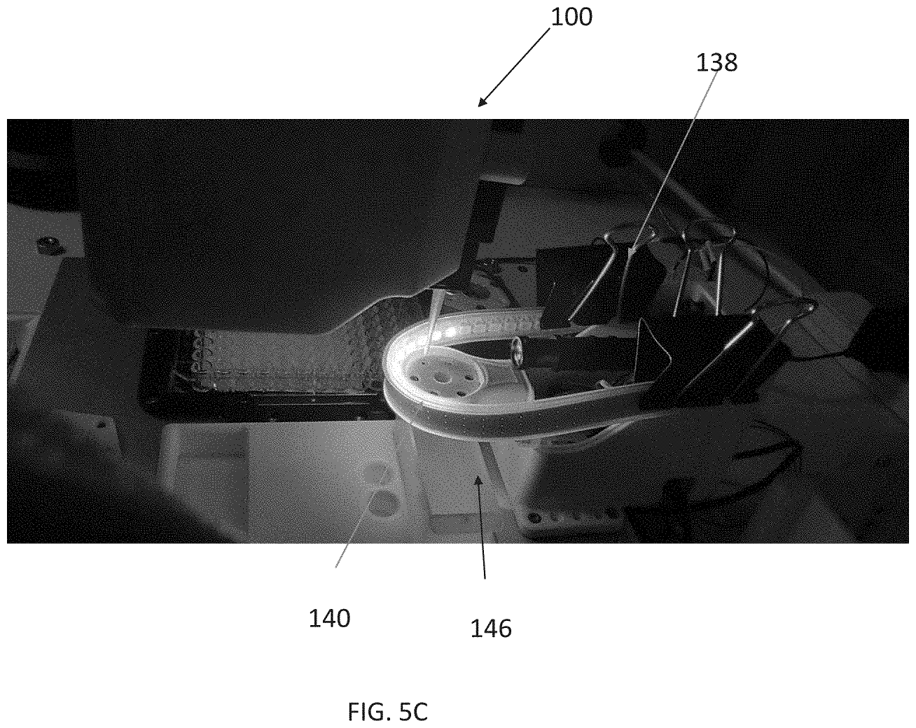

Still referring to FIG. 4, an air knife 140 may be integrated into or otherwise coupled to the print stage 136. Referring to FIGS. 5A-5C, the air knife 140 may generally include a body 141 defining an air knife aperture 142. The body 141 further defines an air-flow opening 144 extending circumferentially within the air knife aperture 142 such as formed within a sidewall of the body 141 defining the air knife aperture 142. The air knife aperture 142 may be sized so as to receive the tip extension 108 therein. The air-flow opening 144 may be angled to blow air or other gasses (e.g., nitrogen, heated nitrogen, oxygen, or any other suitable fluid) so as to clean or dry an outside surface of the tip extension 108 after retrieving fluid from the fluid reservoir 148, for example. The air knife aperture 142 has an air knife diameter that may be larger than a tip extension diameter of the tip extension 108. In embodiments, the tip extension 108 is inserted into the air knife aperture 142 of the air knife 140; the air-flow opening 144 directs gas into the air knife aperture 142, drying or cleaning the tip extension 108 of any residue. For example, the controller 128, after controlling the tip extension 108 and the impedance-measuring tip 104 to aspirate fluid from the fluid reservoir 148, may position the tip extension 108 into the air knife aperture 142, and activate an air knife blower (not shown) fluidically coupled to the air knife 140 to circulate gas out of the air-flow opening 144. The controller 128 may be configured to activate the air knife blower for a designated period of time (e.g., 10 second or less, 20 second or less, a minutes or less, etc.). In some embodiments, image data from the first camera 112 and/or the second camera 138 may be used to determine if there is wetness or residue on the tip extension 108. In embodiments, it is noted that opposed to a circumferential air-flow opening, one or more discrete air-flow openings may be provided for blowing off the tip extension 108. It is contemplated that the impedance-measuring tip 104 may also get wet from aspirating fluid; accordingly, the air knife 140 may also be used to blow off the impedance-measuring tip 104.

As noted above, the dispensing tool system 200 may further include the second camera 138, such as illustrated in FIG. 4, which may be substantially similar to the first camera 112, unless otherwise noted. For example, the second camera 138 may be a high-powered microscope camera, but coupled to the print stage 136 via any suitable fasteners or couplings. The second camera 138 may be positioned on the print stage 136 to face the dispensing end 105b of the impedance-measuring tip 104 and the tip extension 108. However, it is contemplated that the second camera 138 may capture image data of a number of cells present in one or more of the first volume 109 or the second volume 113. The second camera 138 may be communicatively coupled to the controller 128, which may execute instructions (such as via object recognition software) to determine a number of cells within the first volume 109 and/or the second volume 113. In some embodiments, the controller 128 based on the image data received from the second camera 138 may determine a number of cells from in the second volume 113. In embodiments, the image data may be video data. In some embodiments, the controller 128 may determine the number of cells in the first volume 109 and/or the second volume 113 in real- or near-real-time.

Referring to FIG. 5C, a stage light fixture 146 may be coupled to the print stage 136 via any suitable fasteners or couplings. The stage light fixture 146 may provide light to the impedance-measuring tip 104 and tip extension 108. For example, the robotic arm 10, as controlled by the controller 128 may position the tip extension 108 and/or the impedance-measuring tip 104 in front of the stage light fixture 146 to backlight the impedance-measuring tip 104 and the tip extension 108 relative to the second camera 138. The stage light fixture 146 may include a flexible strip of light-emitting diodes (LEDs) and wrap around the impedance-measuring tip 104 and the tip extension 108, although other configurations are contemplated and possible. In embodiments, the stage light fixture 146 may extend around the air knife 140. The stage light fixture 146 may include an LED, fluorescent bulb, or any other suitable light fixture. The stage light fixture 146 may increase image quality of the first camera 112 and/or the second camera 138 by providing light to the impedance-measuring tip 104 and the tip extension 108. In embodiments, the stage light fixture 146, may be communicatively coupled to the controller 128 such that the controller 128 may execute instructions to operate the stage light fixture 146 during dispensing operations. For example, the stage light fixture 146 may be turned off until otherwise needed. In some embodiments, though not shown, it is contemplated that a similar backlight may be coupled to the tool body 102 to backlight the impedance-measuring tip 104 and the tip extension 108 relative to the first camera 112. In various embodiments, the stage light fixture 146 may be configured to emit different color wavelengths (e.g., red, yellow, blue, green, purple) which may assist in easier detection of cells and/or particles.

In embodiments, the controller 128 via artificial intelligence and machine-learning may be trained (such as via training with a neural network as one or more confirmation models) to identify, via object recognition logic, in the image data (e.g., from the first camera 112 and/or the second camera 138) a cell or particle passing out of the first dispensing outlet 107 and/or positioned within the second volume. For example, using the image data (backlit or not) characteristics of the cell(s) and/or particle(s) may be detected from the image data. Such characteristics may include, but are not limited to, shapes, edges, fluorescence, biomarkers, or the like. For example, fluorescence of cells may differ from surrounding fluid. That is, a particular material (e.g., cell or other particle) may have a particular florescence under a particular color light source. Accordingly, the controller 128 may be trained to identify from the image data, such as based on the florescence, biomarkers, and/or other characteristics, the presence of a cell or particle, a number of cells or particles, a size of the cell(s) or particle(s), a material type (e.g., bacteria, cell type, organoid, etc.), or the like, either positioned within the second volume or passing from the first volume to the second volume. This identification allows the controller 128 to execute confirmation logic to confirm that the determinations made based on the impedance measurements are accurate. In various embodiments, such confirmation determinations may be used as training data for training the one or more identification models noted above, accordingly, data may be continuously integrated for more accurate determinations. In some embodiments, a user, using a user input device, may annotate image data for confirmation or training of the one or more identification models.

A method for dispensing cells is generally depicted with respect to FIGS. 6A-6D. The method may be performed using the end effector assembly 100 and/or the dispensing tool system 200 described above. With respect to FIGS. 6A and 6B, as an initial step, of the present disclosure may include submerging at least a portion of the tip extension 108 of the impedance-measuring tip 104 into the fluid reservoir 148 and aspirating the first fluid volume 151 into the first volume 109 of the impedance-measuring tip 104 through the tip extension 108. For example, the controller 128 may operate the pump 115 to aspirate the first fluid volume 151 in the fluid reservoir 148. In embodiments, the first fluid volume 151 may be initially aspirated an amount less than a full volume of both the first volume 109 and the second volume 113. For example, prior to fully filling the first volume 109, the tip extension 108 may be withdrawn from the fluid reservoir 148, and air may be aspirated to clear the second volume 113, thereby ensuring zero cells are present in the second volume 113. It is contemplated such may be confirmed via image data of the first camera 112 or the second camera 138. With respect to FIG. 6C, the method may further include dispensing a portion of the first fluid volume 151 from the first dispensing outlet 107 into the second volume 113 of the tip extension 108 as the second fluid volume 152. As described above, the method may further include measuring the change in impedance of the second fluid volume 152 with the impedance-measuring sensor 110, as the second fluid volume 152 passes from the first volume 109 of the impedance-measuring tip 104 to the second volume 113 of the tip extension 108. With reference to FIGS. 6C and 6D, the method may further include translating the impedance-measuring tip 104 over the print stage 136 and dispensing the second fluid volume 152 into the receptacle 120 through the second dispensing outlet 101 of tip extension 108. For example, the dispensing step may occur in response to the controller 128 determining the desired number of cells and/or the desired cell material is within the second volume 113 of the tip extension 108. In embodiments, the method may include confirming either by a user reviewing the image data from the first camera 112 and/or the second camera 138, or by the controller 128 confirming by performing object recognition on the image data to confirm the cell number and/or material within the second volume 113 prior to dispensing.

As described above, and prior to dispensing, the method may further include drying or cleaning the impedance-measuring tip 104, including the tip extension 108, with the air knife 140. For example, after submerging a portion of the impedance-measuring tip 104, including for example the tip extension 108, into the fluid reservoir 148, there may be some of the working fluid residue on an exterior surface of the tip extension 108. The method may require precise measurements of the second fluid volume 152 to be dispensed through the second dispensing outlet 101 of the tip extension 108. Thus, it is desirable that the exterior surface of the tip extension 108 be free of the working fluid or any residue thereof. Accordingly, the method may include drying the exterior surface of the tip extension 108 by inserting the tip extension 108 into the air knife aperture 142, running the gas from the air-flow opening 144 over the tip extension 108, and withdrawing the tip extension 108 from the air knife aperture 142.

In some embodiments, where it is determined an undesired number of cells or cell material is positioned within the second volume 113, the method may further include dispensing, such as automatically dispensing, by the controller 128 operating the pump 115, the second fluid volume 152 into the waste receptacle 150.

In embodiments, the method may further include storing a history of cells dispensed in a desired well or the waste receptacle 150 on a memory of the controller 128 as noted above.

Embodiments can be described with reference to the following numbered clauses, with preferred features laid out in the dependent clauses:

1. An end effector assembly comprising: a tool body configured to be mounted to a robotic arm; an impedance-measuring tip coupled to the tool body, the impedance-measuring tip defining a first volume configured to receive a fluid and a first dispensing outlet for dispensing the fluid from the first volume, the impedance-measuring tip comprising an impedance-measuring sensor configured to output a signal indicative of change in impedance of the fluid advanced out of the first dispensing outlet, the change in impedance being indicative of cells or other objects passing from the first volume out of the first dispensing outlet; a tip extension fluidically coupled to the impedance-measuring tip, the tip extension defining a second volume for receiving the fluid from the first dispensing outlet; a camera coupled to the tool body and configured to capture image data of the second volume, wherein the image data captures at least a visual representation of the number of cells or other objects in the second volume; and a pump coupled to the impedance-measuring tip and operable to dispense the fluid from the first volume into the second volume and from the second volume into a receptacle via a second dispensing opening formed in the tip extension.

2. The end effector assembly of clause 1, further comprising: a controller communicatively coupled to the impedance-measuring sensor, wherein the controller is configured to determine a number of cells or other objects present in the second volume based on the signal from the impedance-measuring sensor.

3. The end effector assembly of any preceding clause, further comprising: a controller communicatively coupled to the impedance-measuring sensor and the camera, wherein the controller is configured to: determine a number of cells or other objects present in the second volume based on the signal from the impedance-measuring sensor; and confirm the number of cells or other objects present in the second volume based on the image data.

4. The end effector of any preceding clause, wherein the camera is communicatively coupled to a display displaying the image data.

5. The end effector assembly of any preceding clause, wherein the pump is further configured to draw the fluid into the first volume through the second volume.

6. The end effector assembly of any preceding clause, further comprising a tool light fixture coupled to the tool body and configured to backlight the second volume relative to the camera.

7. The end effector assembly of any preceding clause, wherein the impedance-measuring tip has a fluid volume capacity of up to about 200 microliters.

8. The end effector assembly of any preceding clause, wherein the tip extension has a fluid volume capacity of up to about 2.0 microliters.

9. The end effector assembly of any preceding clause, wherein the pump is operable to dispense the fluid from the first volume into the second volume and from the second volume into a waste well via the second dispensing opening formed in the tip extension.

10. A dispensing tool system, the system comprising: a tool body configured to be mounted to a robotic arm; an impedance-measuring tip coupled to the tool body, the impedance-measuring tip defining a first volume configured to receive a fluid and a first dispensing outlet for dispensing the fluid from the first volume, the impedance-measuring tip comprising an impedance-measuring sensor configured to output a signal indicative of a change in impedance of the fluid advanced out of the first dispensing outlet, the change in impedance being indicative of cells or other objects passing from the first volume out of the first dispensing outlet; a tip extension fluidically coupled to the impedance-measuring tip, the tip extension defining a second volume for receiving the fluid from the first dispensing outlet; a camera coupled to the tool body and configured to capture image data of the second volume, wherein the image data captures at least a visual representation of the number of cells or other objects in the second volume; a print stage comprising a substrate, the substrate comprising a receptacle that receives the second volume; a second camera coupled to the print stage and configured to capture the image data of the second volume; and a pump coupled to the impedance-measuring tip and operable to dispense the fluid from the first volume into the second volume and from the second volume into the receptacle via a second dispensing opening formed in the tip extension.

11. The dispensing tool system of clause 10 further comprising an air knife comprising a body defining: at least one aperture, wherein the at least one aperture has an air knife diameter, wherein the air knife diameter is larger than a tip extension diameter of the tip extension; and an air-flow opening extending circumferentially with the at least one aperture so as to direct air flow into the at least one aperture.

12. The dispensing tool system of clause 11, wherein the air knife is coupled to the print stage.

13. The dispensing tool system of any of clauses 10-12, wherein the camera and the second camera are communicatively coupled to a display displaying the image data.

14. The dispensing tool system of any of clauses 10-13, further comprising a stage light fixture coupled to the print stage and configured to backlight the second volume relative to the camera and/or the second camera.

15. The dispensing tool system of any preceding clause, further comprising a tool light fixture coupled to the tool body and configured to backlight the second volume relative to the camera and the second camera.

16. The dispensing tool system of any preceding clause, wherein the robotic arm is a multi-axis robotic arm.

17. A method for dispensing cells, the method comprising: submerging at least a portion of a tip extension fluidically coupled to an impedance-measuring tip into a fluid reservoir, the impedance-measuring tip defining a first volume configured to receive a fluid and a first dispensing outlet for dispensing the fluid from the first volume, the impedance-measuring tip comprising an impedance-measuring sensor configured to output a signal indicative of a change in impedance of the fluid advanced out of the first dispensing outlet, the change in impedance being indicative of cells or other objects passing from the first volume out of the first dispensing outlet, the tip extension defining a second volume; aspirating a first volume of fluid into the first volume of the impedance-measuring tip through the tip extension; dispensing a portion of the first volume of fluid from the first dispensing outlet into the second volume of the tip extension as a second volume of fluid; measuring the change in impedance of the fluid advanced out of the first dispensing outlet and into the second volume with the impedance-measuring sensor; determining with a controller communicatively coupled to the impedance-measuring sensor a number of cells or other objects within the second volume of fluid; translating the impedance-measuring tip over a print stage; and dispensing the second volume of fluid from the second volume into a receptacle when the numbers of cells within the second fluid volume matches a desired number of cells or other objects.

18. The method of clause 17, wherein the desired number of cells or other objects is a single cell.

19. The method of clauses 17 or 18, further comprising confirming the number of cells or other objects in the second volume of fluid by capturing image data of the second volume with a camera communicatively to a display.

20. The method of any of clauses 17-19, further comprising drying the tip extension with an air knife

21. The method of any of clauses 17-20, further comprising training one or more identification models to determining one or more characteristics of the cells or particles, wherein the step of determining with the controller a number of cells or other objects within the second fluid volume, comprises executing the one or more identification models.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. An end effector assembly comprising:
a tool body configured to be mounted to a robotic arm;
an impedance-measuring tip coupled to the tool body, the impedance-measuring tip defining a first volume configured to receive a fluid and a first dispensing outlet for dispensing the fluid from the first volume, the impedance-measuring tip comprising an impedance-measuring sensor configured to output a signal indicative of a change in impedance of the fluid advanced out of the first dispensing outlet, the change in impedance being indicative of cells or other objects passing from the first volume out of the first dispensing outlet;
a tip extension fluidically coupled to the impedance-measuring tip, the tip extension defining a second volume for receiving the fluid from the first dispensing outlet;
a camera coupled to the tool body and configured to capture image data of the second volume, wherein the image data captures at least a visual representation of the cells or other objects in the second volume; and
a pump coupled to the impedance-measuring tip and operable to dispense the fluid from the first volume into the second volume and from the second volume into a receptacle via a second dispensing opening formed in the tip extension.

2. The end effector assembly of claim 1, further comprising:
a controller communicatively coupled to the impedance-measuring sensor, wherein the controller is configured to determine a number of cells or other objects present in the second volume based on the signal from the impedance-measuring sensor.

3. The end effector assembly of claim 1, further comprising:
a controller communicatively coupled to the impedance-measuring sensor and the camera, wherein the controller is configured to:
determine a number of cells or other objects present in the second volume based on the signal from the impedance-measuring sensor; and
confirm the number of cells or other objects present in the second volume based on the image data.

4. The end effector assembly of claim 1, wherein the camera is communicatively coupled to a display displaying the image data.

5. The end effector assembly of claim 1, wherein the pump is further configured to draw the fluid into the first volume through the second volume.

6. The end effector assembly of claim 1, further comprising a tool light fixture coupled to the tool body and configured to backlight the second volume relative to the camera.

7. The end effector assembly of claim 1, wherein the impedance-measuring tip has a fluid volume capacity of up to about 200 microliters.

8. The end effector assembly of claim 1, wherein the tip extension has a fluid volume capacity of up to about 2.0 microliters.

9. The end effector assembly of claim 1, wherein the pump is operable to dispense the fluid from the first volume into the second volume and from the second volume into a waste well via the second dispensing opening formed in the tip extension.

10. A dispensing tool system, the system comprising:
a tool body configured to be mounted to a robotic arm;
an impedance-measuring tip coupled to the tool body, the impedance-measuring tip defining a first volume configured to receive a fluid and a first dispensing outlet for dispensing the fluid from the first volume, the impedance-measuring tip comprising an impedance-measuring sensor configured to output a signal indicative of a change in impedance of the fluid advanced out of the first dispensing outlet, the change in impedance being indicative of cells or other objects passing from the first volume out of the first dispensing outlet;
a tip extension fluidically coupled to the impedance-measuring tip, the tip extension defining a second volume for receiving the fluid from the first dispensing outlet;
a camera coupled to the tool body and configured to capture image data of the second volume, wherein the image data captures at least a visual representation of the cells or other objects in the second volume;
a print stage comprising a substrate, the substrate comprising a receptacle that receives the second volume;
a second camera coupled to the print stage and configured to capture the image data of the second volume; and
a pump coupled to the impedance-measuring tip and operable to dispense the fluid from the first volume into the second volume and from the second volume into the receptacle via a second dispensing opening formed in the tip extension.

11. The dispensing tool system of claim 10 further comprising an air knife comprising a body defining:
at least one aperture, wherein the at least one aperture has an air knife diameter, wherein the air knife diameter is larger than a tip extension diameter of the tip extension; and
an air-flow opening extending circumferentially with the at least one aperture so as to direct air flow into the at least one aperture.

12. The dispensing tool system of claim 11, wherein the air knife is coupled to the print stage.

13. The dispensing tool system of claim 10, wherein the camera and the second camera are communicatively coupled to a display displaying the image data.

14. The dispensing tool system of claim 10, further comprising a stage light fixture coupled to the print stage and configured to backlight the second volume relative to the camera and the second camera.

15. The dispensing tool system of claim 14, further comprising a tool light fixture coupled to the tool body and configured to backlight the second volume relative to the camera and the second camera.

16. The dispensing tool system of claim 10, wherein the robotic arm is a multi-axis robotic arm.

17. A method for dispensing cells, the method comprising:
submerging at least a portion of a tip extension fluidically coupled to an impedance-measuring tip into a fluid reservoir, the impedance-measuring tip defining a first volume configured to receive a fluid and a first dispensing outlet for dispensing the fluid from the first volume, the impedance-measuring tip comprising an impedance-measuring sensor configured to output a signal indicative of a change in impedance of the fluid advanced out of the first dispensing outlet, the change in impedance being indicative of cells or other objects passing from the first volume out of the first dispensing outlet, the tip extension defining a second volume;
aspirating a first fluid volume into the first volume of the impedance-measuring tip through the tip extension;
dispensing a portion of the first fluid volume from the first dispensing outlet into the second volume of the tip extension as a second fluid volume;
measuring the change in impedance of the fluid advanced out of the first dispensing outlet and into the second volume with the impedance-measuring sensor;
determining with a controller communicatively coupled to the impedance-measuring sensor a number of cells or other objects within the second fluid volume;
translating the impedance-measuring tip over a print stage; and
dispensing the second fluid volume from the second volume into a receptacle when the numbers of cells within the second fluid volume matches a desired number of cells or other objects.

18. The method of claim 17, wherein the desired number of cells or other objects is a single cell.

19. The method according to claim 17, further comprising confirming the number of cells or other objects in the second fluid volume by capturing image data of the second volume with a camera communicatively to a display.

20. The method of claim 17, further comprising drying the tip extension with an air knife

21. The method of claim 17, further comprising training one or more identification models to determining one or more characteristics of the cells or particles, wherein the step of determining with the controller a number of cells or other objects within the second fluid volume, comprises executing the one or more identification models.
